(19) United States
(12) Patent Application Publication
(10) Pub. No.: US 2024/0245774 A1
(43) Pub. Date: Jul. 25, 2024

(54) COMPOSITION AND A PHOTODYNAMIC THERAPEUTIC METHOD TO SHORTEN INFECTIVITY PERIOD AND TO INDUCE SUSTAINED HUMORAL AND CELLULAR T-CELL RESPONSES AGAINST A TARGETED ANTIGEN IN INFECTED INDIVIDUALS

(71) Applicant: ONDINE INTERNATIONAL AG, Zug (CH)

(72) Inventors: Nicolas G. Loebel, Woodinville, WA (US); Alejandro Fernandez-Montero, Pamplona (ES); José Maria Argemi Ballbe, Pamplona (ES); Carolyn Cross, Vancouver (CA)

(21) Appl. No.: 18/418,839

(22) Filed: Jan. 22, 2024

Related U.S. Application Data
(60) Provisional application No. 63/441,125, filed on Jan. 25, 2023.

(51) Int. Cl. A61K 41/00; A61K 31/155; A61K 31/5415; A61P 31/14

(52) U.S. Cl. CPC A61K 41/0057; A61K 31/155; A61K 31/5415; A61P 31/14

(57) ABSTRACT

The present invention provides a photodynamic therapeutic method to shorten the infectivity period of an infection caused by a targeted microorganism comprising: applying a photosensitizer on a targeted body treatment area; and applying light to the targeted body treatment area at a wavelength absorbed by the photosensitizer. The present invention also provides a method to induce a sustained humoral and cellular T-Cell responses against an antigen of a targeted microorganism comprising: applying a photosensitizer on a targeted body treatment area; and applying light to the targeted body treatment area at a wavelength absorbed by the photosensitizer.

### FIELD OF INVENTION

The present invention provides a composition and a photodynamic therapeutic method to shorten infectivity period and to induce sustained humoral and cellular T-Cell responses against a targeted antigen in infected individuals.

### BACKGROUND OF THE INVENTION

The pandemic of coronavirus 2019 (“COVID-19”) infections has resulted in over 590 million infections and at least 6.44 million deaths worldwide. It has affected humans of all ages and in all walks of life across the globe, with a clearly defined upper respiratory tract viral pathogenesis often followed by lower respiratory tract severe infections in some cases and in a variant and subject-dependent fashion, evolving to acute respiratory failure and death.

COVID-19 infections are caused by the severe acute respiratory syndrome coronavirus 2 (“SARS-CoV-2”) and its variants (e.g., U.K., South Africa, California, Alpha, Delta, Omicron, etc.). Thanks to the rapid development of vaccines a broad population immunity in most countries, at least against lethal variants, has been achieved. Nevertheless, as seen with the most efficacious mRNA vaccines, despite achieving a significant reduction in morbidity and mortality, all current interventions are failing to prevent the onset of new pandemic waves of Omicron variant (BA.1, BA.1.1, BA.2, etc.), and it is uncertain what protection will result, if any, against future viral genotypes. Thus, public health authorities are advising health authorities and the public not to consider the battle against the worst pandemic of our era to be over.

The present invention provides a composition and a photodynamic therapeutic method to shorten infectivity period of an individual infected with a disease-causing microorganism such as SARS-CoV-2 or other viruses.

The present invention also provides a composition and a photodynamic therapeutic method to induce sustained humoral and cellular T-cell responses against antigens of such disease-causing microorganism.

### BRIEF DESCRIPTION OF THE DRAWINGS

The features and inventive aspects of the present invention will become more apparent upon reading the following detailed description, claims, and drawings, of which the following is a brief description:

FIG. 1 is a graphical depiction of a portion of the photosensitizing chlorhexidine composition application process of the present invention;

FIG. 2 is a graphical depiction of the anterior illumination light application process of the present invention using a light applicator;

FIG. 3 is a graphical depiction of the posterior illumination light application process of the present invention using the light applicator shown in FIG. 2;

FIG. 4 is a graphical depiction of the anterior illumination light application process of the present invention using another light applicator;

FIG. 5 is a graphical depiction of the posterior illumination light application process of the present invention using the light applicator shown in FIG. 4; and

FIG. 6 is a flow chart of the patient recruitment process for the clinical study detailed in Example I;

FIG. 7 is a table of the patients' baseline characteristics for the clinical study detailed in Example I;

FIGS. 8A-C show the results of infectivity assays and RT-PCR on nasopharyngeal swabs conducted for the clinical study detailed in Example I;

FIG. 9 is a table of the risk of infectivity capacity according to PCR test delta-Ct 3 and 7 days after start of treatment for the clinical study detailed in Example I;

FIG. 10 is a table of the risk of having a transmission capacity at 7 days depending on the antigen test and different Cts cut-off points in the PCR test treatment for the clinical study detailed in Example I;

FIG. 11A-D show the results of humoral immunity assays conducted for the clinical study detailed in Example I;

FIG. 12 is a table of the antibody assays for the clinical study detailed in Example I;

FIG. 13 is a table of T-cell immunity assays for the clinical study detailed in Example I;

FIG. 14A-E show the results of cellular immunity assays conducted for the clinical study detailed in Example I; and

FIG. 15 is a table of the COVID19 symptoms detected during the clinical study detailed in Example I.

### DESCRIPTION OF THE PREFERRED EMBODIMENT

Photodynamic therapy fundamentally involves the use of light energy to activate one or more photosensitizers of a photosensitizing composition so that those photosensitizers can then either pass energy on directly to a substrate/target (type I reaction) or can interact with molecular oxygen to produce reactive oxygen species (type II reaction). These reactions have been shown to kill disease causing microorganisms likely primarily via lipid peroxidation, membrane damage, and damage to intracellular components. Accordingly, photodynamic therapy has been used to treat patients suffering from various infectious diseases caused by viruses, bacteria, and fungi (hereinafter collectively referred to as “targeted microorganism’).

The combination of a low concentration of chlorhexidine with a photosensitizer has previously shown to increase the effects of photodynamic therapy in eliminating a targeted microorganism. See U.S. Pat. Nos. 8,247,406B2 and 8,618,091B2.

Early in the COVID-19 pandemic, single-cell RNA sequencing studies in healthy human subjects showed a particularly high expression of viral entry factors, with the most affected target being the ACE2 receptor located in the goblet and multi-ciliated cells of the nasal epithelium, although these proteins were also expressed in lower but still significant levels in other tissues, especially the multi-ciliated airway epithelium. While these data were useful to understand both the rapid spread and predominantly pneumonic phenotype of severe COVID-19, they also underlined the importance of the nose as the entry site and potential primary replication reservoir in the first few days after SARS-CoV-2 inoculation of the host.

The photodynamic therapeutic method of the present invention (hereinafter referred to as “PDT”) comprising activating a photosensitizer (e.g., methylene blue) on a targeted body treatment area (e.g., the anterior nares) by light has been shown to (i) shorten the infectivity period of an infection (e.g., COVID-19) caused by a targeted microorganism (e.g., SARS-CoV-2); and (ii) induce a sustained humoral and cellular T-cell responses against the antigen of such targeted microorganism (e.g., SARS-CoV-2).

The PDT of the present invention is comprised of the following processes. First, applicators (e.g., pre-soaked swabs) containing a photosensitizing composition (hereinafter referred as “PC”) are provided. The PC includes a photosensitizer (e.g., phenothiazinium), and optionally a low concentration of chlorhexidine. The photosensitizer of the PC used in the clinical study discussed in Example I was the phenothiazinium methylene blue, at a concentration of 0.01% total weight (% wt).

Other photosensitizers that effect both Type I and Type II photoreactions, where Type I reactions produce electron abstraction redox-type reactions upon the application of light and Type II reactions produce singlet oxygen (via molecular oxygen) upon the application of light, are also suitable for use in the PC. Preferred phenothiaziniums for the PC include not only methylene blue, but also toluidine blue, and those discussed in U.S. patent Publication No. 2004-0147508. Another preferred photosensitizer for the PC is indocyanine green. The present invention also contemplates the use of two or more photosensitizers, such as methylene blue and toluidine blue or the like. The photosensitizers mentioned above are examples and are not intended to limit the scope of the present invention in any way.

In certain embodiments, the photosensitizer can be tetrapyrroles or derivatives thereof such as porphyrins, chlorins, bacteriochlorins, phthalocyanines, naphthalocyanines, texaphyrins, verdins, purpurins or pheophorbides, phenothiazines, etc., such as those described in U.S. Pat. Nos. 6,211,335; 6,583,117; and 6,607,522 and U.S. patent Publication No. 2003-0180224. For example, suitable classes of compounds that may be used as the photosensitizer include pyrrole derived macrocyclic compounds, porphyrins, chlorins, bacteriochlorins, isobacteriochlorins, phthalocyanines, naphthalocyanines, porphycenes, porphycyanines, pentaphyrins, sapphyrins, benzochlorins, chlorophylls, azaporphyrins, the metabolic porphyrinic precusor 5-amino levulinic acid, synthetic diporphyrins and dichlorins, phenyl-substituted tetraphenyl porphyrins, indium chloride methyl pyropheophorbide, 3,1-meso tetrakis (opropionamido phenyl) porphyrin, verdins, purpurins, zinc naphthalocyanines, anthracenediones, anthrapyrazoles, aminoanthraquinone, phenoxazine dyes, chlorins, benzoporphyrin derivatives, sulfonated aluminum phthalocyanine, tetrasulfonated derivative, sulfonated aluminum naphthalocyanines, chloroaluminum sulfonated phthalocyanine, phenothiazine derivatives, chalcogenapyrylium dyes, cationic selena and tellurapyrylium derivatives, ring-substituted cationic phthalocyanines, pheophorbide alpha, hydroporphyrins, phthalocyanines, hematoporphyrin, protoporphyrin, uroporphyrin III, coproporphyrin III, protoporphyrin IX, 5-amino levulinic acid, pyrromethane boron difluorides, indocyanine green, zinc phthalocyanine, dihematoporphyrin, benzoporphyrin derivatives, carotenoporphyrins, hematoporphyrin and porphyrin derivatives, rose bengal, bacteriochlorin A, epigallocatechin, epicatechin derivatives, hypocrellin B, urocanic acid, indoleacrylic acid, rhodium complexes, etiobenzochlorins, octaethylbenzochlorins, sulfonated Pc-naphthalocyanine, silicon naphthalocyanines, chloroaluminum sulfonated phthalocyanine, phthalocyanine derivatives, iminium salt benzochlorins, and other iminium salt complexes, DNA-binding fluorochromes, psoralens, acridine compounds, suprofen, tiaprofenic acid, non-steroidal anti-inflammatory drugs, methylpheophorbide-a-(hexyl-ether), and other pheophorbides, furocoumarin hydroperoxides, Victoria blue BO, methylene blue, toluidine blue, porphycene compounds, and combination thereof.

The photosensitizer may be present in the PC in any suitable amounts. Examples are between about 0.005% wt and to about 1% wt, between about 0.01% wt to about 0.1% wt, between about 0.01% wt to about 0.05% wt, and no more than about 1% wt. The percentage of total weight (% wt) can also be converted to percentage of total weight to volume (% w/v) or percentage of total volume to volume (% v/v). For the purpose of this specification, the concentration of photosensitizer can be expressed either in % wt, % w/v, or % v/v and such expression of concentration is intended to include its equivalences (e.g., if expressed in % wt, it is intended include the equivalent concentration measured in % w/v and % v/v). The term “about” as used herein in this specification shall mean +/−20% of the stated.

The concentration of chlorhexidine used in the PC for the study was chlorhexidine gluconate at a concentration of 0.25% wt. Other forms of chlorhexidine such as chlorhexidine digluconate, chlorhexidine dihydrochloride, chlorhexidine diacetate, etc. are also suitable for use in the PC. Exemplary suitable concentrations are about 1% wt; about 0.5% wt; about 0.25% wt; about 0.125% wt; between about 0.125% wt and about 1% wt; between about 0.125% wt and about 0.5% wt; between about 0.2% wt and about 0.3% wt; between about 0.125% wt and about 0.3% wt; a range that is less than about 1% wt but more than about 0.1% wt; a range that is less than about 0.8% wt but more than about 0.1% wt. It is preferred that the chlorhexidine is provided at a concentration that reduces and/or eliminates potential irritation and sensitivity to host tissues at the treatment site. This reduction and/or elimination of potential irritation and sensitivity is especially helpful when the host tissues at the treatment area are sensitive tissues such as the nasal mucosa.

The PC further optionally includes a pharmaceutically acceptable carrier. The pharmaceutically acceptable carrier is a diluent, adjuvant, excipient, or vehicle with which the other components (e.g., the photosensitizer and the chlorhexidine, etc.) of the PC are administered. The pharmaceutically acceptable carrier is preferably approved by a regulatory agency of the Federal or a state government or listed in the U.S. Pharmacopeia or other generally recognized pharmacopeia for use in animals, and more particularly in humans. The pharmaceutically acceptable carriers are preferably sterile liquids. Examples of the pharmaceutically acceptable carriers include but are not limited to water, saline solution, dextrose solution, glycerol solution, phosphate buffered saline solution, etc.

It is further preferred that the pharmaceutically acceptable carrier, when combined with the photosensitizer and the chlorhexidine, allows the PC to have a viscosity low enough to flow into the targeted treatment site while also having a viscosity high enough to maintain the PC within the targeted treatment site. Further compositions that become liquid after application to the targeted treatment site are contemplated such as those that melt or go into solution in the targeted treatment site. Alternately, the PC may gel after application to the treatment site as a liquid, as a non-limiting example by using reverse-phase polymeric substrates; this would permit the PC to cover the treatment site effectively, while also maintaining the composition in the treatment site.

PDT may further include an optional cleaning of the user's nostrils, especially the nasal passages contained within the upper nostrils. During the study, each non-infected user blew his/her nose using a tissue. This nostril cleaning process can also be achieved via swabbing each nostril with a clean swab. Moreover, such clean swab can be soaked in a cleaning solution such as water, saline, or the like prior to its use in the nostril cleaning process.

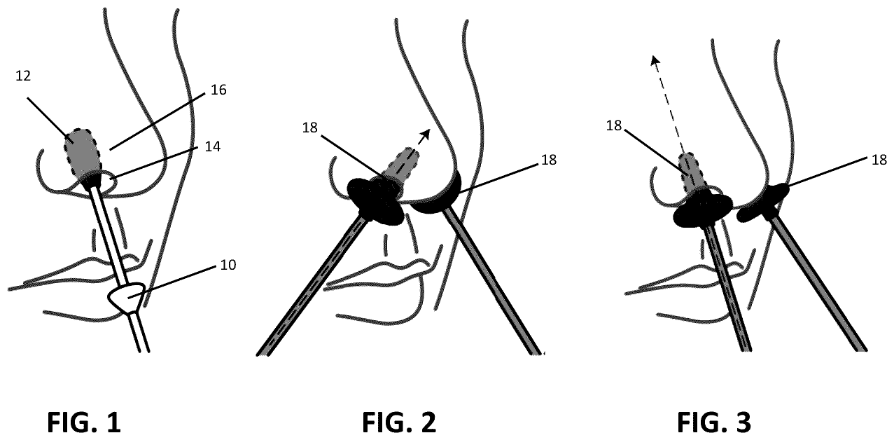

Once the nostrils are cleaned, the PDT further includes applying the PC by inserting the applicator 10 containing the PC 12 into one of the user's nostrils 14 and swabbing the nasal treatment site 16 inside the nostril 14 in order to deposit the PC 12 uniformly and generously onto the nasal treatment site 16 as shown in FIG. 1. It is preferred that the applicator 10 is disposable and single use. Depending on the size(s) of the user's nostrils, it is preferred that the applicator 10 is inserted at least about 2 to 3 cm deep into each nostril 14. The nasal treatment site 16 is defined as the interior surface of the nostril 14 including, but is not limited to, all areas below the turbinate and including the lower turbinate area. It is preferred that special care is taken to get good coverage in the most anterior nasal pocket area (i.e., the tip of the nose) of the nasal treatment site 16. The PCC application process is repeated in the user's other nostril (not shown in FIG. 1) in order to deposit the PC 12 uniformly and generously onto the nasal treatment site 16 within this second nostril.

After the PC application processes for both nostrils have been completed, the PDT further includes the light application process. The light application process is comprised of applying light to each of the nasal treatment site in a wavelength that can be absorbed by the photosensitizer(s) contained in the PC. This light application process can be achieved using any art-disclosed suitable methods and light sources such as laser diodes, light emitting diodes, infrared, and enhanced pulsed light beam or combinations thereof.

Prior to the application of light to the nasal treatment sites, the PC is optionally placed into contact with the nasal treatment site for a short period of time ranging from less than 15 to more than 120 seconds, acting as a pre-incubation period.

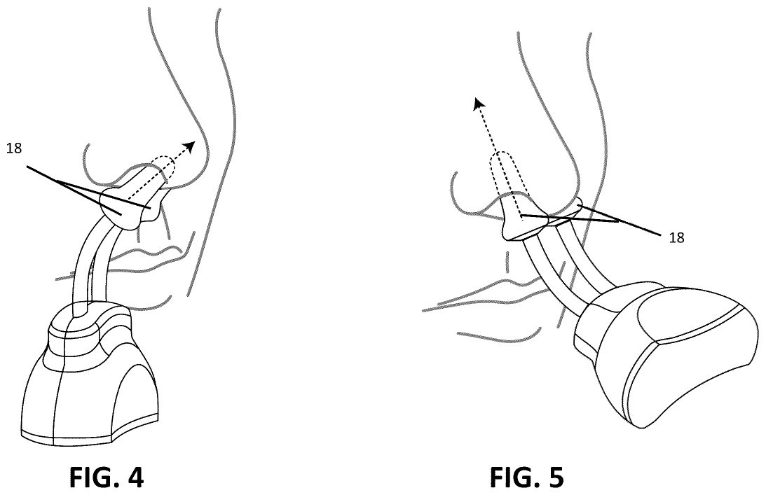

In one embodiment of the PDT, the light application process is conducted twice: once for posterior illumination of the nasal treatment sites as shown in FIGS. 3 and 5 and once for the anterior illumination of the nasal treatment sites 16 as shown in FIGS. 2 and 4. For the posterior illumination of the nasal treatment sites, the PDM involves inserting light diffuser tips 18 straight into the user's nostrils 14 as shown in FIGS. 3 and 5 by orienting the light diffuser tips 18 to point towards the back of the user's head for a 2-minute illumination cycle at a wavelength matched to the primary absorption wavelength of the sensitizer chosen for use. Once this posterior illumination is completed, the PDT may optionally include removing the light diffusing tips 18 from the user's nostrils 14 and repeating the PC application processes for both of the user's nostrils 14 in preparation for the anterior illumination of the nasal treatment sites 16. For the anterior illumination of the nasal treatment sites 16, the PDT involves inserting light diffusing tips 18 into the user's nostrils 14 as shown in FIGS. 2 and 4 by orienting the light diffuser tips 18 anteriorly (away from the face) and medially toward the distal tip of the user's nose for a 2-minute illumination cycle at a wavelength matched to the primary absorption wavelength of the sensitizer chosen for use. Upon completion of the anterior illumination, the light diffuser tips 18 are removed from the user's nostrils 14. In a second embodiment, the PDM involves inserting light diffuser tips 18 straight into the user's nostrils 14 as shown in FIGS. 3 and 5 by orienting the light diffuser tips 18 to point towards the back of the user's head for a 2-minute illumination cycle at a wavelength matched to the primary absorption wavelength of the sensitizer chosen for use, removing the light diffuser tips 18, re-swabbing the PC into the nostrils and re-inserting the light diffuser tips 18 in an identical fashion for a second treatment cycle. For the study, the light diffuser tips 18 were powered by a light source capable of generating sufficient optical power at a wavelength between 630 nm and 690 nm for the illumination cycles. An example of a suitable light source is a Class 1 laser device consisting of 2 channels of 700 mW. As noted above, other suitable light sources can also be used to accomplish PDT.

Depending on the photosensitizer concentration and the power of the light emitting device(s), the application of light to treatment sites may only require a short period of time such as from about 15 seconds to less than about 5 minutes, preferably from about 1 minutes to about 3 minutes, and from about 2 minutes to about 4 minutes. The light energy per area provided during each cycle of application of light is preferred to range from about 2 J/cm² to about 45 J/cm², preferably at about 18 J/cm² to about 36 J/cm. It is preferred that multiple light treatments (e.g., about 2 to about 10, about 3 to about 5, etc.) are applied to each treatment site thereby resulting in a total accumulated light energy applied to the treatment site that can be substantially higher than the light energy provided during each cycle.

The PDT can be repeated multiple times (e.g., about 2 to about 5, etc.) if desired. It is preferred that the applications of light to the treatment sites do not cause physiological damage to the host tissues at and/or surrounding the treatment sites.

### EXAMPLE I

A single centre, randomized, placebo-controlled, single-blind, clinical trial study was conducted in a university hospital in Northern Spain. Participants were recruited from the University of Navarra COVID19 Safe Campus Program and the Clinica Universidad de Navarra COVID19 Surveillance program. Both programs were established in 2020 during the pandemic, in order to perform active surveillance after release of the first population lockdown in Spain and limit new viral spread within the University Campus and Hospital. The programs were based on both random and contact-traced individual testing using either rapid-antigen or PCR testing.

Inclusion criteria for the study required patients to be 18 years old or older and present within 48 h of determination of SARS-CoV-2 positivity based on real-time PCR (less than 27 cycles) or rapid antigen test, along with asymptomatic or only mild illness levels. All concomitant medications other than angiotensin receptor blockers or immunosuppressant agents were allowed. All patients consented to receive either the non-painful nasal light illumination process or a placebo equivalent.

Patients with severe comorbidities were excluded. Individuals who reported inability to tolerate insertion of the light illuminator due to oronasal size, shape, or anatomical variants, those with known allergic reactions to components of the nasal decolonization treatment including methylene blue or chlorhexidine gluconate, those with COVID-19 illness that was moderate or severe in nature, and those who were unable to attend the necessary follow-up appointments were excluded from the study.

Participants were randomly assigned by blocks to either the control group or the treatment group (1:1) using a computer program after inclusion in the trial. The person who performed the randomization also had other tasks assigned to him/her, such as assessing the inclusion/exclusion criteria, receiving the informed consent from the patient and completing the baseline and follow-up questionnaires. This person did not apply the treatment to any of the groups and did not collect any biological samples.

The control treatment was performed using saline solution and a sham treatment with a switched-off laser illuminator device, with the patient blinded to the intervention using polarizing glasses. The treatment was not blind to the investigator. Placebo-treated participants followed the same timing and cycle protocol as the intervention group.

The PDT of the present invention was performed using the CE-marked Steriwave™ Nasal Photodisinfection System (NPS, SW4000, Ondine Biomedical Inc, Vancouver, BC, Canada). Briefly, an NPS is a Class II Medical Device that includes a power source (“Light Source”), a Nasal Light Illuminator (NLI) which consists of a single-use, double-way, laser-conducting, injection-molded nasal applicator, and a methylene blue formulation at a concentration of 0.01% total weight (% wt) and chlorhexidine gluconate at a concentration of 0.25% total weight (PC) approved in Canada and Europe for the decolonization of potentially pathogenic microorganisms from the anterior nasal passages. The topically applied PC binds microbial cell wall components, and the red light (having wavelengths between 664 nm to 670) is absorbed by the photosensitizer molecules, producing reactive oxygen species (ROS) that are responsible for the lethal microbial cell wall disruption. Please note that the red light can also have wavelengths between 630 nm to 690 nm, 650 nm to 680 nm, and 660 nm to 670 nm). Moreover, light having wavelengths between 500 nm to 800 nm can also be used to perform the present invention.

Nasopharyngeal (NP) swabs were performed at baseline before treatment, after the 3-day treatment, and on the 7^{th} day after the beginning of the treatment. NP specimens were collected in Universal Transport Media (UTM, Copan) and used for immediate RNA extraction (TANBead® Nucleic Acid Extraction Kit) and RT-PCR (Cepheid Xpert Xpress SARS-CoV-2, genes E and N) and were kept in UTM for in vitro infectivity assays in Biosafety Level 3 facilities. An aliquot of total nucleic acid extracted from baseline NP swabs was used for SARS-CoV2 sequencing.

The NPS was used by previously trained healthcare professionals. The procedure was initiated by swabbing the PC inside the patient's anterior nares, including the nostril and the nasal passages. The operator then connected the NLI to the power source and inserted the nasal double end of the NLI into the patient's nostrils. In this protocol, the Light Source was turned on, and a 4-minute illumination cycle was applied for 3 cycles using two new additional PC after each illumination cycle, to ensure equal performance. This 4 min×3 scheme was repeated on day 2 and 3. In total each patient was treated for 36 min in nine 4-minute cycles. To avoid self-contamination NLI cables were labelled with “L” for the left nostril and “R” for the right nostril. Each day a new set of NLI was used to avoid self-contamination. The subjects were asked to blow their nose prior to treatment, but local treatments were not allowed. The placebo control arm included an application of saline and introduction of the turned-off PDT device, following the protocol as in the intervention group, for the three consecutive days.

The primary outcome of the study was the reduction of infectivity after 3 days of treatment. Before the beginning of the enrolment, considering the evidence pointing to the permanence of PCR positivity results due to nucleic acid remanent and not to viable virus, in vitro infectivity assays with Vero-E6 cells were preferred to RT-PCR as endpoint, although both techniques were performed in each sample. Secondary outcome was safety and reduction of infectivity at other time points (7 and 14 days after the beginning of treatment). Biological correlates of the study included the analysis of the patients' immunogenicity against Nucleocapsid, Spike and total SARS-Cov2 genome at 10 and 20 weeks after treatment and the genomic sequencing of the specimens, as described below.

Patients were asked about their symptoms present 24 hours prior to the baseline visit, day 3 and day 7. The symptoms collected were sore throat, chills, new or worsening cough, respiratory distress, chest tightness, temperature over 38° Celsius, fatigue, muscle pain, loss of smell, loss of taste, headache, gastrointestinal symptoms, difficulty sleeping, general malaise, nasal and congestion. They were collected on a four-category scale (absent, mild, moderate, or severe).

To evaluate safety, any immediate local treatment effects was be determined as well as any delayed local effects. The severity of these and the likelihood of treatment-relatedness, as measured subjectively by the investigator, were also noted. These questions were asked immediately after treatment and at the interview at each subsequent visit.

Other variables were also collected such as sex, age, complete primary covid vaccination, booster dose of covid vaccine, previous covid infection, weight, height and vital signs such as body temperature, heart rate, systolic and diastolic blood pressure and blood oxygen saturation.

The sample size calculation was performed based on the differences found in the pre and post intervention delta-Ct of the preliminary invitro study after 4 and 8 minutes of applying PDT on the infected sample. At 99% power only a small sample size was needed to demonstrate microbiological efficacy (6 in each group). It was assumed that the subjects would have a wide range of ages and comorbidities, making assumptions about their clinical course and viral load decline difficult. We also knew that there was a spontaneous decline in viral load in our study population (i.e., mostly vaccinated and otherwise healthy individuals). In addition, a 10% dropout rate was considered. With all this, a sample size of 100 patients was proposed, 50 in each group. Due to the rapid fade of the sixth COVID19 pandemic wave in Spain (December 2021-February 2022) we could not reach the complete sample size.

To compare the different quantitative variables between the control group and the intervention group, a Mann-Whitney U test was performed for those that did not follow normality and a Student's t test for those that did. Medians and interquartile ranges and mean and standard deviations were calculated, respectively. A Chi² test was performed for qualitative variables.

In the infectivity trial analyses, a multiple linear regression model was performed, and Beta coefficients and their respective 95% confidence intervals (95% Cl) were calculated to verify the treatment effect. To assess the ability to reduce contagion in diagnostic tests, multiadjusted logistic regression models were performed and Odds Ratios (OR) and their 95% Cl were estimated. Both models were adjusted for: sex, age, number of initial symptoms, COVID vaccine, COVID booster dose and previous SARS-CoV-2 infection.

All p values presented are two-tailed. Prism software (GraphPad Software, San Diego, CA) and STATA13.0 were used for statistical analysis.

In vitro Infectivity Assay: Confluent monolayers of Vero-E6 cells were passaged to confluence on 96 well plates and infected with the samples from all patients previously diluted 1:2 with infection medium (Minimum Essential Medium—MEM with 0.2% BSA 0.2%, 2 mM glutamine and 20 mM Hepes) and incubated for 4 h at 37° C. After removing the inoculum, Eagle's MEM with 10% foetal bovine serum and antibiotics was added to each well of infected cells. Non-infected cells were used as negative controls. After 72 h, cells were collected, lysed using Dynabeads™ MyOne™ Silane beads and the amount of SARS-CoV-2 genomes was analysed by RT-PCR (Real-Time Fluorescent RT-PCR Kit for Detecting SARS-CoV-2, BGI, genes ORF1ab and Human β-actin). These values are expressed as 2^(−ΔCt)*1000.

Specific Anti-SARS-Cov2 humoral and cellular responses: To analyse the serological response of the participants, a blood draw was performed 10 and 20 weeks after entry into the clinical trial.

Anti-SARS-CoV-2 antibody detection was performed using four different commercial chemiluminescence tests. First, quantification of total antibodies (IgG+IgM) against the receptor binding domain (RBD) of SARS-CoV-2 spike (S) protein was performed, using Elecsys® Anti-SARS-CoV-2 S (Roche Diagnostics, Germany) test in the cobas e601 platform. Second, qualitative detection of total antibodies (IgG+IgM) against viral nucleocapsid (Anti-N) was done using Elecsys® Anti-SARS-CoV-2 test (Roche Diagnostics, Germany). Third, Anti-SARS-CoV-2 specific IgG against nucleocapsid and spike proteins was detected using COVID-19 VIRCLIA® IgG Monotest (Vircell SL, Spain). Fourth, Anti-SARS-CoV-2 IgM+IgA against nucleocapsid and spike proteins were detected using COVID-19 VIRCLIA® IgM+IgA Monotest (Vircell SL, Spain). Serum samples were previously inactivated at 56° C. for 30 minutes. The interpretation of the different immunoassays was done as recommended by each manufacturer.

Cell-mediated immune response to SARS-CoV-2 was measured using QuantiFERON® SARS-CoV-2 Starter and Extended Sets (QIAGEN, USA). The Starter Set included specific peptides from the spike antigen (S1, S2, RBD subdomains) to evaluate CD4 (Ag1 tube) and CD4+CD8 (Ag2 tube) T cells immune responses. The Extended Set contained additional specific peptides from the full genome of SARS-CoV-2 (S, N and M domains) to study a complete specific CD4 and CD8 T cell-mediated immune responses (Ag3 tube). After inoculation, the tubes were incubated for 20-24 hours at 37° C., and subsequently IFN-γ concentrations were measured in plasma by QuantiFERON® ELISA (QIAGEN, USA). Samples were considered reactive when any tube showed IFN-γ production, following stimulation, above 0.15 IU/mL.

Sequencing analysis of COVID19 variants: Libraries were prepared from swab samples using a commercial kit (COVIDSeq Assay, Illumina) and sequenced using a NextSeq2000 (Illumina). Analyses were performed with Kraken (Illumina). The phylogenetic tree was performed with Nextclade.org.

Invitro preliminary studies to assess photodynamic effect on SARS-CoV2: Prior to the current study, the efficacy of PDT on SARS-CoV2 survival ex vivo was unknown. For this reason, an in vitro preliminary study was conducted at the Biosafety Level 3 (BSL3) facility of the Gene Therapy Division of Centre for Applied Medical Research (CIMA), University of Navarra. At this end, a nasopharyngeal swab sample with high load of SARS-CoV-2 from a severe COVID-19 patient was cultured in confluent Vero-E6 cells and the supernatant was collected at 72 h post-inoculation. The virus was then filtered and titrated using a lysis plate assay using Vero-E6 cell monolayers, resulting in a titre of 4.3×107 plaque-forming units per millilitre (PFU/ml). Twenty uL of viral solutions containing 1×104 and 1×105 PFU of SARS-CoV-2 were prepared using phosphate-buffered saline (PBS) solution. Each sample was then mixed with 180 μl of photosensitizer formulation (PF) and added to small plastic reservoir simulating a human nostril (nasal tip reservoir). As controls, virus samples were mixed with 180 μl of PBS, without PF. We then applied to each sample the NLS for 0, 4, or 8 minutes, slightly shaking the NLI every two minutes to allow reoxygenation of samples. Then the NLI was removed, and each sample was collected and analysed by RT-PCR to quantify the amount of SARS-CoV-2 genomes. Viral RNA was extracted from each sample using Dynabeads™ MyOne™ Silane beads (ThermoFisher) and quantified by RT-qPCR with oligonucleotides specific for SARS-CoV-2 nucleocapsid gene. The results obtained confirmed the increasing SARS-CoV2 viral nucleic acid destruction when treating 104 or 105 Plaque-forming Units (PFU) with 4 to 8 min of PDT exposure. We did not retain this data suggestive of in vivo efficacy, but just a proof of the deleterious effect of PDT on viable viral particles.

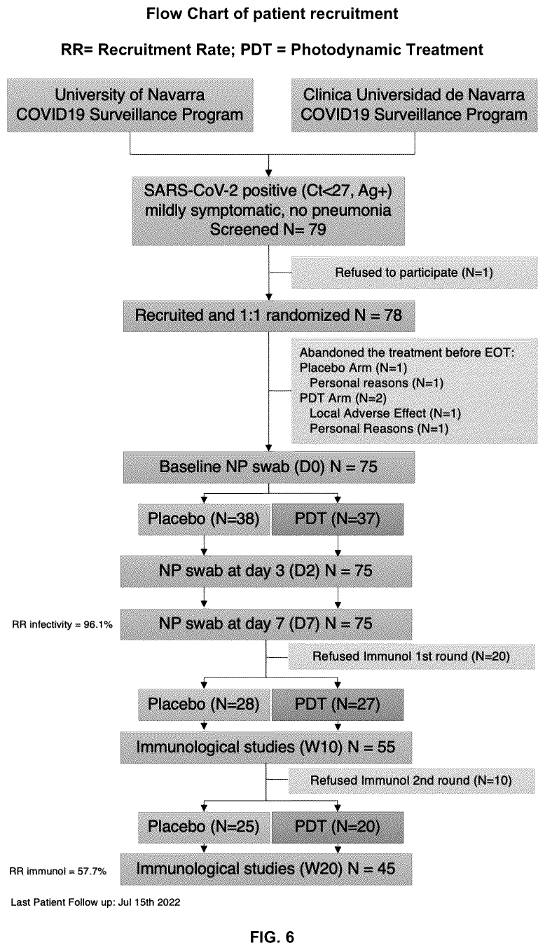

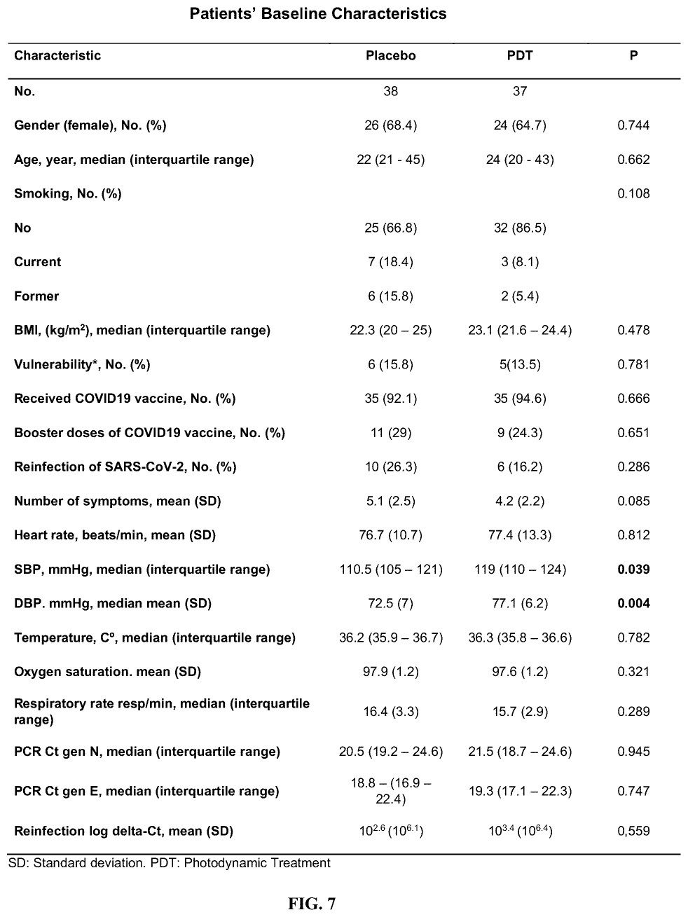

Results: Seventy-nine patients were screened between Dec. 21, 2021, and Feb. 15, 2022, during the 6^{th} COVID-19 pandemic wave in Spain. One patient was excluded for not meeting the criteria. Of the 78 patients randomized 1:1, two patients abandoned the treatment group (one for local irritation, the other for personal reasons), and one patient in the placebo group (personal reasons). Seventy-five patients completed the study were included in the analyses. The retention rate was thus 96.1%. See FIG. 6.

The population consisted of mostly young adults, with mild symptoms, naïve to natural SARS-CoV2 infection (79%) with a complete course of vaccination (i.e., two doses of mRNA-based vaccines) (93%), and an expected high viral load See FIG. 7. No significant differences were observed between the intervention and the placebo group, except in the measured blood pressure, although the effect and the absolute values were deemed not clinically relevant.

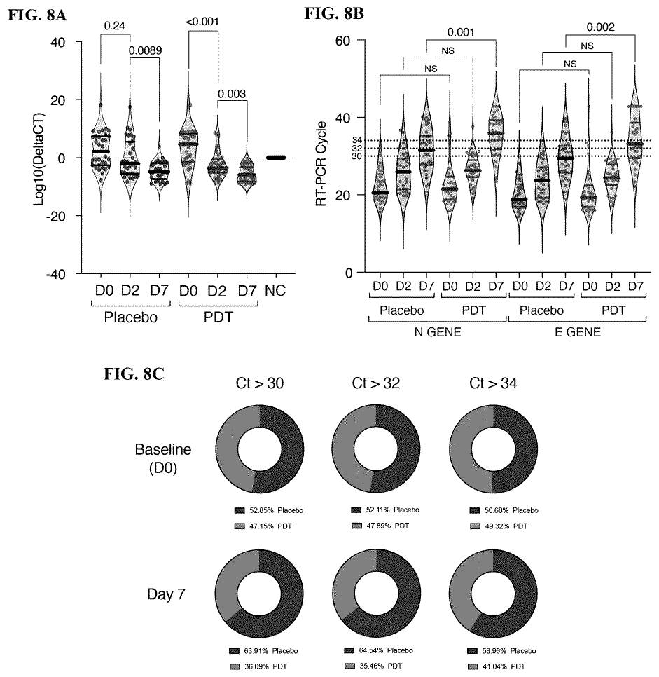

Treated patients showed a significant decrease in infectivity from baseline to 3 days after treatment (p<0.0001), while placebo patients did not (p=0.24) See FIG. 8A. Both placebo group and treated patients had a significant decrease in infectivity from the 3rd to the 7th day, although the decrease was more intense in the treated group (p=0.003) than in the placebo group (p=0.0089) (FIG. 8A).

FIGS. 8A-C show the results of infectivity assays and RT-PCR on Nasopharyngeal Swabs. For FIG. 8A, the Subjects recruited in the clinical study underwent nasopharyngeal swabs at baseline (Day 0), at day 2 and at day 0. The sample was collected in Viral transport medium and was subsequently used for the analysis of infectivity on VeroE6 cells. Seventy-two hours after infection, the supernatant was tested for the presence and quantity of SARS-CoV2 using RT-PCR for Orf1b gene and human beta actin. Referring to FIG. 8A, data is expressed in log10 of the delta between beta actin and Orf1b gene fluorescence threshold cycle. A Wilcoxon test for paired data was used for comparing baseline to D2 and D2 to D7. For FIG. 8B, RT-PCR of E and N genes of SARS-CoV2 from nasopharyngeal swab samples immediately after collection. Referring to FIG. 8B, data is expressed in fluorescence threshold cycle. A Wilcoxon test for paired data was used for comparing baseline to D2 and D2 to D7. C. Percentage of “positive” and “negative” individuals in each of the two groups at baseline, D2 and D7, depending on which cycle threshold was chosen.

In the multi-adjusted linear regression model shows a protective effect of treatment at 3 days with a mean β coefficient of −812 (95% Cl −478660-−1.3, p<0.05) and a trend at 7 days with a mean β coefficient of −9.3 (95% Cl −269.1-3.1, p NS). See FIG. 9 which is a table showing the risk of infectivity capacity according to PCR test delta-Ct 3 and 7 days after start of treatment.

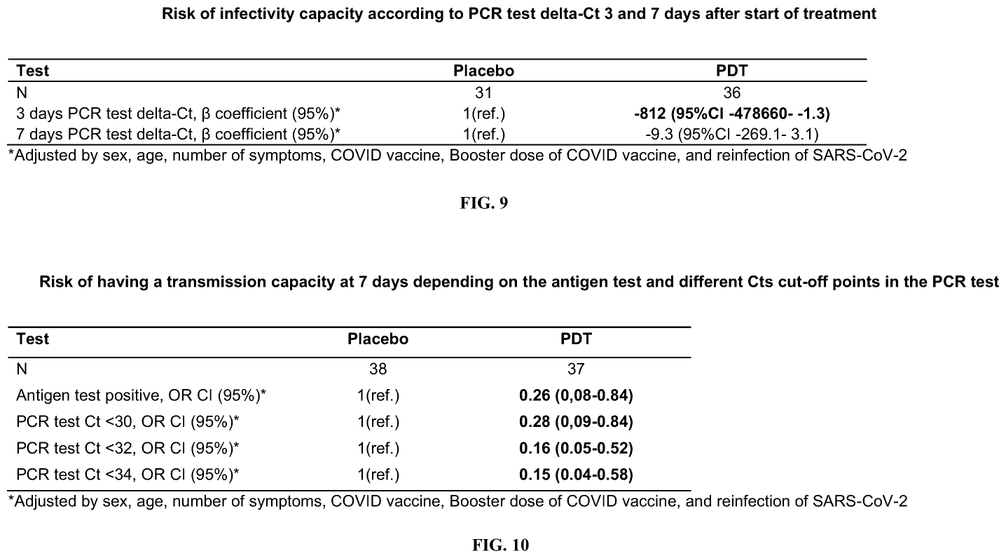

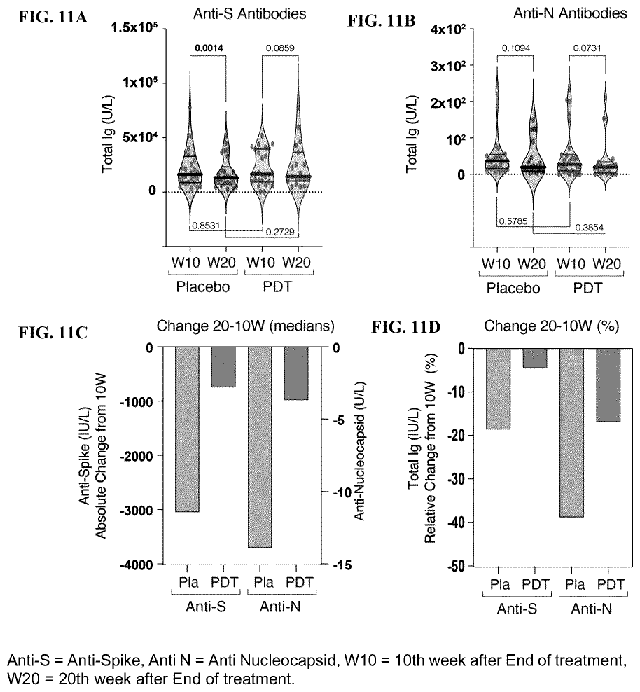

Along the study period, both treatment and placebo groups had increased mean PCR cycle, although treated patients showed a significant difference in RT-PCR cycle of both E and N genes 7 days after the beginning of the treatment (FIG. 8B). When analysing the probability of being PCR negative at 7 days, nasal PDT showed protection against PCR positivity at 7 days with Odds Ratio of 0.16 (95% Cl: 0.05-0.52) and 0.15 (95% Cl: 0.04-0.58), using thresholds of 32 or 34 cycles respectively. See FIG. 10; FIG. 8C.

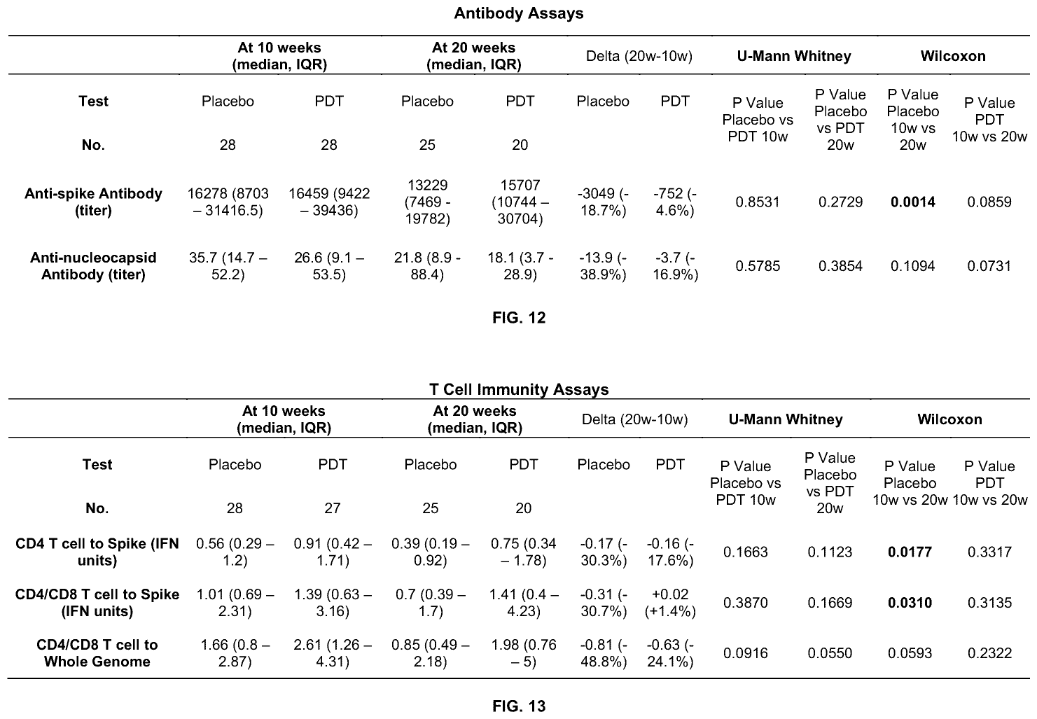

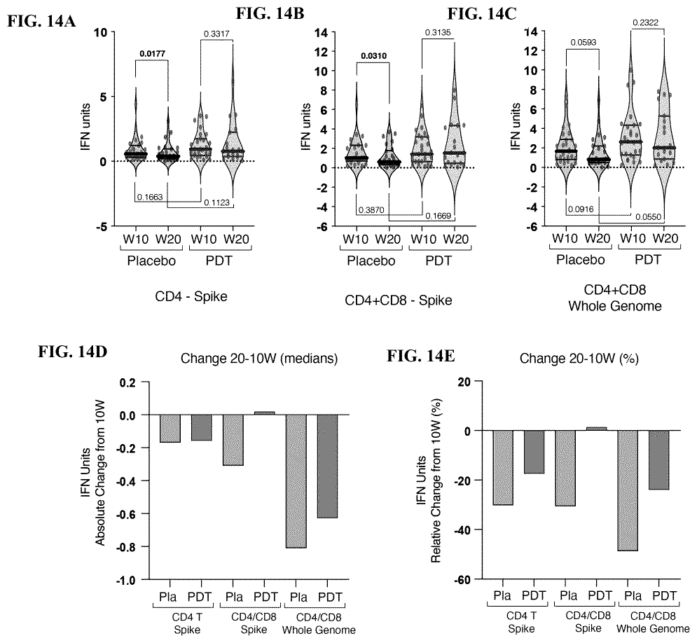

Referring to FIGS. 11A-D, the results of humoral immunity assays are shown. Patients were followed up after intranasal PDT treatment for 20 weeks. On week 10 and week 20, a sample of plasma and peripheral CD4 and CD8 T lymphocyte was collected. Detection of plasma total anti-Spike antibodies as shown in FIG. 11A. Detection of plasma total anti-Nucleocapsid antibodies are shown in FIG. 11B. Change of median levels at 20 weeks when compared to 10 weeks are shown in FIG. 11C. Percentage of change of median antibody levels at 20 weeks with respect to 10 weeks as shown in FIG. 11D. The results of the antibody assays and cell immunity assays are shown in respectively in FIG. 12 and FIG. 13. Referring to FIGS. 14A-E, the results of cellular immunity assays are shown. Quantiferon assay was used to analyse the specific T-cell responses against specific purified peptides from the spike antigen (S1, S2, RBD subdomains) either using isolated CD4 (as shown in FIG. 14A) or a combination of CD4/CD8 (as shown in FIG. 14B). T cells from placebo and PDT-treated subjects, or against additional specific peptides from the full genome of SARS-CoV-2 (S, N and M domains) testing both CD4 and CD8 responses (as shown in FIG. 14C). Change of median values of IFN units at 20 weeks when compared to 10 weeks for the three assays are shown in FIG. 14D. Percentage of change of median IFN units at 20 weeks when compared to 10 weeks are shown in FIG. 14E.

Antibody quantification at 10 and 20 weeks after treatment showed no differences in the production of anti-spike (FIG. 11A) and anti-nucleocapsid (FIG. 11B) antibodies, between control and PDT arms (FIG. 12). There was a significant decay in Anti-Spike antibodies at 20 weeks in the placebo group (p<0.001), but not in the PDT group (p=0.1336) (FIG. 11A). The measurement of interferon (IFN) synthesis by CD4 T cells after exposure to SARSCoV2 spike antigen revealed lower median IFN units in the placebo group when compared to PDT both at 10 and at 20 weeks (FIG. 13; FIG. 14A). Combined CD4 and CD8 T cell responses against the Spike protein were also trending higher in PDT-treated individuals with medians at 20 weeks almost doubling that of Placebo (p=0.0971) (FIG. 13; FIG. 14B). When CD4 and CD8 cells were exposed to the products of the whole genome of SARS-CoV2, increased T cell responses were observed in the PDT group, both at 10 weeks, and at 20 weeks (p=0.0293) (Table 5, FIG. 4C). In both time points, the decay of specific T cell immunity against SARS-CoV2 from 10 to 20 weeks was significant in the control group, while the level of SARS-CoV2-induced IFN production from 10 to 20 weeks was similar in PDT-treated individuals (FIG. 14C).

The sequencing of COVID19 RNA was successful in 72 patients (96% of the population). The results indicate that 97.2% of patients had the Omicron 21K variant (B.1.1.529 or BA.1) with only 2 exceptions: one patient with Omicron 21L (BA.2) and one colonized by Delta 21J variant.

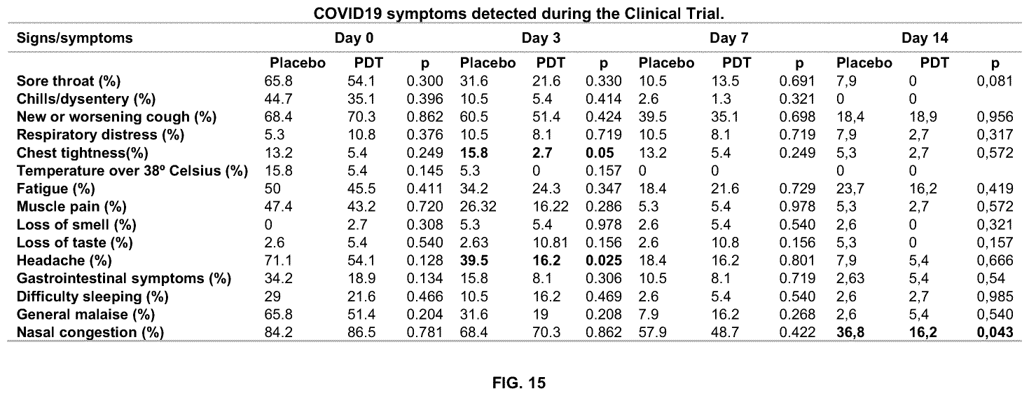

When analysing the progression of symptoms at day 3 and 7 after the start of treatment, a significant decrease in the proportion of chest tightness and headache in the PDT-treated group when compared to the placebo group was observed on day 3. See FIG. 15 which provides a listing of the COVID19 symptoms detected during the clinical trial.

Regarding safety of the intervention, 32 patients (5 in the control group and 27 in the intervention group) reported a total of 53 mild adverse events. One patient of the intervention group (2.7%) dropped out of the study due to intense itching in the nostrils after first day of treatment.

COVID-19 pandemics has led to a dramatic challenge to the health and economy of individuals, societies, and entire countries worldwide. Several variants of SARS-CoV2 have been sequentially causing viral endemic waves of different clinical and biological profile from the end of 2019 to current days, with clinical outcomes ranging from severe COVID-19 pneumonia and death to completely asymptomatic cases.

In early stages of SARS-CoV2 infection, especially in mildly symptomatic carriers of less lethal variants such as Omicron, the local decolonization of nasal passages, the most likely entry and initial replication site of the virus, could be relevant for the inhibition of viral spread, in different clinical and non-clinical settings. We believe our study is the first prospective randomized trial exploring the tolerance and relevance of nasal decolonization in mildly symptomatic SARS-CoV2 infected individuals.

The primary endpoint of the trial (i.e., the reduction of infectivity measured with in vitro infectivity assay with nasopharyngeal swab samples) was achieved. We show the effect of nasal PDT in mildly symptomatic otherwise healthy individuals with high viral load (Ct<26 and/or antigen test positivity at recruitment). The treatment consisted of three consecutive day, 12 min-treatment of methylene blue PDT.

Interestingly, although the treatment with PDT was applied only to the nasal passages, the treatment reduced SARS-CoV-2 colonization of the entire upper respiratory tract when compared to placebo. This could be due to the nasal mucosa as the main site of viral replication in the case of Omicron variant and/or vaccination status. This result strengthens the hypothesis of the relevance of nasal decolonization in a disease that could develop to a systemic stage in a few days. The effects of PDT were observed as soon as 3 days after treatment using in vitro infectivity assay using NP swabs. Although considered a less reliable readout, RT-PCR cycles in NP samples were higher in the treated group at 7 days, a further sign of the durable effect of nasal decolonization. Consequently, PDT treatment had an important protective effect for avoiding PCR positivity at 7 days. There were no serious adverse effects during treatment.

We observed a sharp decrease in viral load in our entire population (placebo and PDT) at day 3 and 7, which could damper the magnitude of the effect of PDT. A potential explanation is that our population was mostly vaccinated (93%): indeed, vaccination is associated with faster decline in viral RNA in patients with SARS-CoV-2 infection. On the other hand, almost all patients were infected predominantly by Omicron variant (21K, BA.1) (97.2%) with a favourable biology when compared to other variants. It is possible that low aggressive variants could be less persistent in the upper airway when compared to alpha or delta variants. These two facts, the high percentage of vaccinated individuals and the rapid decline of viral load, underscores the efficacy of PDT for eliminating infectious viral particles, being the decay in the treated group significantly higher than the placebo as soon as three days on treatment.

Importantly and unexpectedly, we show that the treatment with PDT produces a profound and durable impact on SARS-CoV2-specific T lymphocyte-mediated immunity. We have shown almost double median IFN unit production by CD4 and CD8 T lymphocytes in individuals treated with nasal PDT. Most importantly, while a decay between the 10th and the 20th week post-treatment was significant in placebo-treated individuals, all T-Cell responses evaluated were sustained after PDT treatments. We believe this is the first evidence of PDT-enhanced immunization against SARS-CoV2.

The sustained effect on immunity caused by the application of PDT provides us with novel immunization strategies. For example, we can use a combination of photosensitizer with antigens aiming at provoking a photochemical internalization of the antigens into Antigen Presenting Cells (APC), which facilitates their adjuvant-free crosspriming to CD8 T lymphocytes.

The present invention further includes local treatments of PDT to reduce the viral load in patients with early or mildly symptomatic viral infections that are not caused by SARS-CoV-2 (i.e., caused by other viruses). The present invention also includes local treatments of PDT to reduce the bacterial load in patients with early or mildly symptomatic bacterial infections. Finally, the present invention includes local treatments of PDT to reduce the fungal load in patients with early or mildly symptomatic fungal infections.

The local treatments of PDT discussed in this application can be applied to anybody.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes.

What is claimed is:

1. A photodynamic therapeutic method to shorten the infectivity period of an infection caused by a targeted microorganism comprising: applying a photosensitizing composition comprising a photosensitizer on a targeted body treatment area; and applying light to the targeted body treatment area.

2. The method of claim 1, wherein the targeted body treatment area is the anterior nares.

3. The method of claim 2, wherein the targeted microorganism is selected from a group consisting of a coronavirus, a SARS-CoV-2, a variant of SARS-CoV-2, and a combination thereof.

4. The method of claim 1 wherein the photosensitizer is phenothiazine.

5. The method of claim 1 wherein the photosensitizer is methylene blue.

6. The method of claim 1 wherein the photosensitizing composition further includes chlorhexidine at a concentration of no more than about 1% wt of the photosensitizing composition.

7. The method of claim 1 wherein the photosensitizer is at a concentration of no more than about 1% wt of the photosensitizing composition.

8. The method of claim 6 wherein the chlorhexidine is at a concentration of between about 0.125% wt and about 1% wt of the photosensitizing composition.

9. The method of claim 6 wherein the photosensitizer is methylene blue at a concentration of 0.01% wt of the photosensitizing composition and the chlorhexidine is at a concentration of 0.25% wt of the photosensitizing composition.

10. The method of claim 1 wherein the light is at a wavelength between 500 nm to 800 nm and is absorbed by the photosensitizer.

11. The method of claim 1 further comprising: cleaning the targeted body treatment area before applying the photosensitizer and the light is applied to the targeted body treatment area for at least two minutes.

12. A method to induce a sustained humoral and cellular T-Cell responses against an antigen of a targeted microorganism comprising: applying a photosensitizing composition comprising a photosensitizer on a targeted body treatment area; and applying light to the targeted body treatment area.

13. The method of claim 12, wherein the targeted body treatment area is the anterior nares.

14. The method of claim 13, wherein the targeted microorganism is selected from a group consisting of a coronavirus, a SARS-CoV-2, a variant of SARS-CoV-2, and a combination thereof.

15. The method of claim 12 wherein the photosensitizing composition further includes chlorhexidine at a concentration of no more than about 1% wt of the photosensitizing composition, and the photosensitizer is a phenothiazine.

16. The method of claim 12 wherein the photosensitizer is methylene blue.

17. The method of claim 12 wherein the photosensitizer is methylene blue at a concentration of 0.01% wt of the photosensitizing composition and the chlorhexidine is at a concentration of 0.25% wt of the photosensitizing composition.

18. A photosensitizing composition for inducing a sustained humoral and cellular T-Cell responses against an antigen of a targeted microorganism comprising: a phenothiazine and chlorhexidine.

19. The composition of claim 16, wherein the phenothiazine is at a concentration of no more than about 1% wt of the photosensitizing composition.

20. The composition of claim 16, wherein the targeted microorganism is selected from a group consisting of a coronavirus, a SARS-CoV-2, a variant of SARS-CoV-2, and a combination thereof.

21. The composition of claim 16, wherein the chlorhexidine is at a concentration of between about 0.125% wt and about 1% wt of the photosensitizing composition.

22. The composition of claim 16 wherein the photosensitizer is methylene blue at a concentration of 0.01% wt of the photosensitizing composition and the chlorhexidine is at a concentration of 0.25% wt of the photosensitizing composition.
